(19) United States
(12) Patent Application Publication
(10) Pub. No.: US 2013/0302293 A1
(43) Pub. Date: Nov. 14, 2013

(54) COMPOSITIONS, CELLS, KITS AND METHODS FOR AUTOLOGOUS STEM CELL THERAPY

(71) Applicant: Keith A. Webster, Key Biscayne, FL (US)

(72) Inventor: Keith A. Webster, Key Biscayne, FL (US)

(21) Appl. No.: 13/884,054

(22) PCT Filed: Nov. 11, 2011
(86) PCT No.: PCT/US11/60306

§ 371 Date: Jul. 29, 2013

Related U.S. Application Data
(60) Provisional application No. 61/412,449, filed on Nov. 11, 2010.

(51) Int. Cl. C12N 15/85; A61K 35/28; C12N 15/86

(52) U.S. Cl. CPC C12N 15/85; C12N 15/86; A61K 35/28
USPC 424/93.21; 435/325; 435/366

(57) ABSTRACT

Described herein are compositions, kits and methods for stimulating angiogenic functions of stem cells and/or progenitor cells having pro-angiogenic potential (e.g., endothelial progenitor cells (EPCs), mesenchymal stem cells (MSCs)) before transplantation (e.g., ex vivo cell therapy) based on the discovery that functional recovery of CD34+ cells from coronary artery disease (CAD) patients is improved by transfection of antagomirs against one or more miRs of a plurality of miRs identified to be over-expressed in cells from CAD patients. Described herein are methods to recover the functions of EPCs isolated from patients with cardiovascular disease (e.g., CAD or peripheral artery disease (PAD)) by bioengineering the cells with antagomirs and/or premirs to specific micro-RNAs. The bioengineered cells can then be used to treat patients with ischemic or ischemic-related disease (e.g., CAD or PAD) by autologous stem cell therapy.

### CROSS-REFERENCE TO RELATED APPLICATIONS

This application claims the benefit of Provisional Application Ser. No. 61/412,449 filed Nov. 11, 2010, which is herein incorporated by reference in its entirety.

### FIELD OF THE INVENTION

The invention relates generally to the fields of medicine, cellular therapy and gene therapy. More particularly, the invention relates to compositions, cells, kits and methods for improving function, survival and proliferation of stem cells and/or progenitor cells (e.g., endothelial progenitor cells (EPCs)) and for treating patients with ischemic disease or ischemic-related disease.

### BACKGROUND

Risk factors associated with atherosclerosis include age, genetics, lifestyle, hypertension and diabetes. There are strong negative correlations between EPC activity, age and the Framingham cardiovascular risk factor score (Castelli WP. Am J Med 1984; 76:4-12; Weinsaft and Edelberg, Am J Geriatr Cardiol 2001; 10:348-354; Kannel and Gordon, Cardiovascular risk factors in the aged the Framingham study. In: Haynes S G, Feinleib M, editors, Epidemiology of Aging. Bethesda, Md.: National Institutes of Health; 1980. 65-98; Lakatta EG. Physiol Rev 1993; 73:413-467; Gerhard et al., Hypertension, 1996; 27:849-853; Zeiher et al., J Clin Invest. 1993; 92:652-662; Gennaro et al., Circulation, 2003; 107:230-233). Recent studies suggest that bone marrow-derived CD34⁺ EPCs support endothelial integrity by repairing injury, and conversely defective EPC function may be a root cause of atherosclerosis. EPCs are characterized by the expression of cell surface antigens CD133, CD34 and KDR. Immature hematopoietic progenitors express all three markers but CD133 is lost after commitment to either hematopoietic or endothelial lineage. EPC markers are moving targets that change as the cells migrate from the bone marrow and home to the vessel wall driven by chemotactic cytokines such as VEGF and SDF-1. The process of vascular regeneration by circulating EPCs is called vasculogenesis and contrasts with angiogenesis that involves local cell activation. In animal models it has been shown that vasculogenesis is responsible for up to 27% of new vessels in granulation tissues (Murayama et al., Experimental Hematology. 2002; 30:967), 45% of tumors (Reyes et al., J Clin Invest. 2002; 109:337-46), 5-20% of regenerating vasculature in hind limb ischemia models and regenerating heart after AMI (van Weel et al., Ann Vasc Surg. 2008 22(4):582-97; Iwasaki et al., Circulation. 2006, 113(10):1311-25). It has been reported that vasculogenesis accounts for almost 100% of revascularized murine ischemic skin flaps. In most of these studies CD34⁺ EPCs are implicated as the functional cell type. EPCs recruited to ischemic tissue provide structural repair and secrete cytokines and growth factors that are protective and promote the proliferation and migration of local cells. Defective CD34⁺ EPC number or function are predicted to reduce the potential for vasculogenesis and promote coronary artery disease (CAD) and impaired tissue recovery from injury.

In the cellular hypothesis of atherosclerosis, progression of the disease is determined not only by the balance of good/bad cholesterols and lipoproteins, but also by the balance of circulating pro- and anti-inflammatory hematopoietic cells and EPCs. Vascular injury within a permissive environment stimulates resident macrophages to degranulate and release pro-inflammatory cytokines that recruit a battery of pro-atherogenic mononuclear cells (CD45⁺, CD14⁺, CD11⁺) from the bone marrow and circulation. These cells infiltrate the endothelium and promote permeabilization, inflammation and atherosclerotic plaque formation. To combat this, Th2-type cytokines and chemokines including IL-3, IL-8, GCSF, VEGF, and SDF-1 are released from the damaged endothelium and recruit protective CD34⁺/KDR⁺ EPCs to sites of injury. The outcome is determined by the severity of the atherogenic environment and the balance between pro- and anti-atherogenic cells. Multiple studies confirm a strong inverse correlation between age, cardiovascular disease and a decline in the numbers and function of EPCs. Colony formation and migration by circulating EPCs is reduced in patients with ischemia, hypercholesterolemia, hypertension and diabetes. CD34⁺ EPCs from patients with ischemic heart disease are defective in the induction of angiogenesis in ischemic limbs. It has even been reported that the overall level of circulating EPCs is a significantly better predictor of vascular reactivity than conventional CAD risk factors. These studies suggest direct relationships between EPC function and the progression of atherosclerosis. Defective CD34⁺ EPCs are predicted to create a permissive environment for atherosclerosis and reduced therapeutic efficacy of autologous bone marrow mononuclear cells (BMMNCs).

The TACT study (Tateishi-Yuyama et al., Lancet. 2002, 360(9331):427-35) reported increased angiographic score, pain-free walking time, ABI and transcutaneous oxygen pressure after i.m. delivery of autologous BMMNCs to 22 peripheral artery disease (PAD) patients at 1-yr follow up. TACT provided immunohistological evidence for endothelial regeneration by stem cell therapy. The TACT study provides evidence that muscles of patients with severe PAD are viable and capable of responding to stem cell therapy. The results of TACT were confirmed in the OPTIPEC trial (Van Huyen et al., Mod Pathol. 2008, 21(7):837-46; Pacilli et al., Ann Vasc Surg. On Line 2009 May 19). Using amputee specimens from OPTIPEC patients, Van Huyen et al reported that active angiogenesis was present in the distal part of the cell-treated ischemic limbs of 3 patients despite requiring amputation. Immunohistochemical staining showed extensive endothelial cell proliferation within the new vessels. The authors concluded that BMMNC cell therapy in patients with PAD induces active, sustained angiogenesis in the ischemic limbs, although this may not prevent amputation in some patients. TACT and OPTIPEC both used autologous stem cells and it is proposed that defective EPCs precluded optimal therapy in both cases.

The MAGIC trial was a randomized trial of BMMNCs mobilized with GCSF and infused into the coronary arteries. MAGIC investigators reported modest but significantly improved LVEF at 6-months but the trial was prematurely halted because of excessive in-stent restenosis (Beitnes et al., Heart. 2009 Oct. 14). In the REPAIR-MI (Dill et al., Am Heart J. 2009 157(3):541-7) and REPAIR-CHF trials, BM or peripheral blood derived progenitor cells were administered by intracoronary infusion into patients with recent or chronic (within 3 months) AMI. REPAIR-MI reported modest (−3%) but significant improvement of LVEF as well as multiple combined end points. Investigators of the TOPOCARE-AMI trial of autologous BMMNC or EPC therapy for AMI also reported modest but significantly improved LVEF (Assmus et al., Circulation. 2002, 106(24):3009-17; Schächinger et al., Trial. J Am Coll Cardiol. 2004, 44(8):1690-9). Similar results were also reported in the BOOST trial (reviewed in (Losordo and Dimmeler, Circulation. 2004; 109:2692-7)). Thus, there is currently a need for improved therapeutic agents and methods for treating ischemic disorders such as PAD and CAD.

### SUMMARY

Described herein are compositions, cells, kits and methods for stimulating angiogenic functions of therapeutic stem or progenitor cells (e.g., EPCs) by modulating micro-RNA (miR) levels in the cells before transplantation into a subject (e.g., human patient). The compositions, cells, kits and methods are based on the discovery that miR profiles of progenitor cells from CAD patients are different from those of healthy volunteers and this correlates with angiogenic and proliferative dysfunction of the cells with CAD origin. Furthermore, additional tests showed that functional recovery of the progenitor cells from CAD patients was improved by transfecting the patient's cells with antagomirs against one or more miRs found to be upregulated in the CAD patients in order to effect suppression of the miRs. Contributions of both down-regulated and up-regulated miRs to cellular dysfunction is implicated by properties of the affected miRs and our observations that miR manipulation can improve function. Described herein are methods to recover the functions of autologous stem cells isolated from patients with ischemia or ischemia-related disease (e.g., CAD or PAD) by bioengineering the cells with antagomirs or premiRs to specific micro-RNAs. The bioengineered cells can then be used to treat patients with, for example, CAD and PAD, by autologous stem cell therapy. Microarray and micro-RNA analyses were performed of CD34+/Lin− cells (putative EPCs) from 5 patients with CAD, 4 age-matched non-CAD patients and 3 healthy volunteers. The arrays revealed 15 micro-RNAs (miRs) that were strongly upregulated (>3-fold) in the CAD group relative to non-CAD or healthy volunteers. In addition, 6 micro-RNAs were selectively downregulated in the CAD group. Upregulated miRs included miR-493, miR-515-5p, miR-196b, miR-1913, miR-520a, miR-1281, miR-373, miR-1978, miR-155, miR-92a, miR-335, miR-1973, miR-21, miR-26a and miR-16. Of these, miRs-16, -21, -26a, -92a, and -155 were identified with properties that are predicted to be especially damaging for pro-angiogenic, progenitor stem cell functions. Mir-16 targets mRNAs encoding VEGFA, CCND1 and CCND2; miR-21 targets bone morphogenic protein receptor 2 (BMPR2); miR-26a targets GSK3μ; miR-92a targets integrin alpha-V and -5 and Akt; and miR-155 is induced by inflammatory cytokines and may modulate the inflammatory response. Most of these targets for downregualted miRs have been confirmed experimentally by RT-PCR. The situation is similar for upregulated miRs including miR-128, miR-720, miR-939, miR-885, miR-154 and miR-373, each of which is potentially a regulator of stem/progenitor cell functions. Mir-128 targets Bmi-1 and ABCC5 thereby regulating p16 and p19 cell cycle inhibitor genes, Bmi-1 knockout in mice results in defects in hematopoiesis, skeletal patterning, and neurological functions (Zhu et al, Clin Cancer Res., September 2011; Epub). Because of elevated miR-128, stem cells/progenitor cells of CAD patients are predicted to have impaired cell cycle regulation. Mir-939 targets TNF-alpha, a cytokine that regulates multiple cell responses including immune responses; TNF-alpha mediates cross talk with NF-kb, MAPKs and apoptosis signaling pathways; cells from CAD patients with elevated miR-939 are predicted to have dysregulated growth and survival properties (Semaan et al, PLoS One 2011; 6(5): e19827). MiRs-885 may target cyclin-dependent kinase 2, NF2, MCM5 and JUN (Afanasyeva et al, Cell Death Differ 18:974-84, 2011; Guled et al, Genes and Chromosomes Cancer, 2009, 48:615-23). Cells from CAD patients with elevated miR-885 are predicted to have dysregulated growth and survival properties. MiR-154 has been linked with acute myeloid leukaemia and may have an important role in regulating gene expression in embryonic stem cells (Su et al, BMC Syst Biol. 2010 Nov. 8; 4:150). MiR-373 modulates the Wnt/β-catenin-signaling pathway with potentially important roles in stem cell division, proliferation and differentiation. Dysregulation of Wnt/β-catenin-signaling by elevated miR-373 in the stem/progenitor cells of CAD patients may severely disrupt angiogenic functions.

In summary, CD34+/Lin− endothelial progenitor cells from CAD patients have multiple dysregulated (up and down-regulated) micro-RNAs that results in parallel dysregulation of multiple genes that regulate survival, proliferative and pro-angiogenic functions. We predict that these changes are responsible for the poor outcome of autologous stem cell therapy for CAD and PAD. We have also observed that the MSCs isolated from bone marrow of patients with CAD have significantly reduced proliferation compared with MSCs from healthy volunteers. We predict that the microenvironment of tissues of patients with CAD, that may be pro-inflammatory, affects the expression of specific micro-RNAs and promotes cellular dysfunction that precludes effective autologous cell therapy of multiple cell types. Manipulation of single or multiple micro-RNAs may reverse some of these defects and regain therapeutic activity. Our finding that inflammation-regulated miR-155 is increased in cells from patients with CAD suggests to us that inflammatory mediators in the serum of patients with CAD (or PAD) is responsible at least in part for the dysregulation of miRs. Therefore we propose that pre-incubation of stem cells with angiogenic potential from patients with CAD, in serum from healthy patients or healthy animals, may reverse some of these defects.

Autologous CD34+ cells are the major cell types used to deliver angiogenic stem cell therapy to patients in the major stem cell clinical trials for CAD and PAD to date. These trials showed only limited efficacy and it is suspected that defective CD34+ cells from the patients due to dysregulated micro-RNAs is a prime reason for this. More recently autologous and syngenic MSCs have been tested with a similarly moderate outcome (Trachtenberg et al, Am Heart J. 2011 March; 161(3):487-93; Lasala et al, Angiology. 2010 August; 61(6):551-6; Lu et al, Diabetes Res Clin Pract. 2011 April; 92(1):26-36). Our experiments show that CD34+ cells from patients with CAD are functionally defective with impaired in vitro tube formation as well as an impaired ability to induce angiogenesis in an ischemic hind limb model of PAD. In further studies, a short-list of miRs were focused on that were considered the most damaging for EPC functions out of this group; these included miRs-16, -21, 92a and 155-. Transfection of these miRs into human umbilical vascular endothelial cells (HUVECs) caused strong inhibition of the functions of these cells, in particular tube formation in Matrigel in response to VEGF was significantly decreased by each miR transfected individually and was abolished when 3 miRs (−16, -21, -92a or -16, -92a, -155) were delivered simultaneously to HUVECs. To see whether functional recovery of CD34+ cells from CAD patients was possible, these cells were transfected with antisense (antagomirs) to miR-16, -21 and -92a and analyzed alongside controls (transfected with miR-1 antagomir) for their ability to form endothelial tubes in a modified HUVEC mixed cell assay. It was found that cells subjected to the combined antagomir treatments and labeled with Dil were able to integrate effectively into HUVEC tubes.

We also studied the effects of miR-155 in similar models. Briefly, microarray and RT-PCR confirmed >5-fold up-regulation of miR-155 in Lin−/CD34+ cells of CAD patients compared with non-CAD (p<0.05). MiR-155 knockdown in HUVECs increased HIF1alpha and CCND1 protein as measured by western blot (p<0.05). MiR-155 over-expression using precursor molecules significantly inhibited tube formation and cell proliferation of HUVECs (both p<0.01). LDH assay revealed an increase in cell death of HUVECs transfected with pre-miR-155 (p<0.05). The results show that miR-155 regulates multiple biological processes including cell differentiation, survival, and cell migration and extrapolate this to suppression of angiogenesis.

In conclusion, our results are consistent with loss of function by overexpression of miRs 16, 21, 92a and 155 in HUVECs, mimicking some of the effects of CAD on endogenous EPCs; conversely, improved function and recovery of EPC-like properties can be achieved by suppression of miRs 16, 21, 92a and 155 by selective antagomirs in CD34+ cells from CAD patients. Whereas the experiments were limited to 4 miR examples it is expected that any and possibly all dysregulated miRs in cells from CAD patients may contribute to defective functions of the cells and appropriate manipulation of miR levels will rectify this and recover function. Thus, compositions and methods to improve functions of autologous cells from CAD patients by using antagomirs to suppress the levels of miRs that are abnormally elevated, methods and compositions to improve functions of autologous cells from CAD patients by using premirs to enhance the levels of miRs that are abnormally decreased, and methods to improve functions of autologous cells from CAD patients by preincubating cells in media designed to reverse the abnormal miR expression profiles seen in freshly isolated cells are all described herein.

Accordingly, described herein is a composition including a plurality of at least one of: stem or somatic cells with pro-angiogenic potential and progenitor cells with pro-angiogenic potential, transduced with at least one nucleic acid encoding at least one of: antagomir to miR-493, antagomir to miR-515-5p, antagomir to miR-196b, antagomir to miR-1913, antagomir to miR-520a, antagomir to miR-1281, antagomir to miR-373, antagomir to miR-1978, antagomir to miR-155, antagomir to miR-92a, antagomir to miR-335, antagomir to miR-1973, antagomir to miR-21, antagomir to miR-26a, antagomir to miR-16, premir of miR-128, premir of miR-720, premir of miR-939, premir of miR-885-3p, premir of miR-154, and premir of miR-373. In the composition, the plurality of cells are isolated from a subject and have increased angiogenic function compared to the same cells (same types of cells) isolated from the subject that were not transduced with the at least one nucleic acid. The plurality of cells can include at least one of: CD34+ EPCs, mesenchymal stem cells (MSCs), Lin− cells from bone marrow or peripheral blood, mononuclear cells from bone marrow or peripheral blood, myofibroblasts, skeletal myocytes, cardiac myocytes, satellite cells, and stem cells. The subject is typically a human and the plurality of cells can be CD34+ EPCs obtained from the human subject. The at least one nucleic acid can encodes antagomirs to miR-16, miR-21 and miR-92a. In another embodiment, the at least one nucleic acid encodes at least one premir of at least one miR selected from the group consisting of: miR-128, miR-720, miR-939, miR-885-3p, miR-154, and miR-373. The at least one nucleic acid can be a viral vector (e.g., a recombinant Adeno-Associated Virus (AAV) vector (rAAV)). The composition can further include a suitable medium for transplantation of the transduced plurality of cells into the subject.

Also described herein is a plurality of at least one of: stem or somatic cells with pro-angiogenic potential and progenitor cells with pro-angiogenic potential, each cell of the plurality of cells transduced with at least one nucleic acid encoding at least one of: antagomir to miR-493, antagomir to miR-515-5p, antagomir to miR-196b, antagomir to miR-1913, antagomir to miR-520a, antagomir to miR-1281, antagomir to miR-373, antagomir to miR-1978, antagomir to miR-155, antagomir to miR-92a, antagomir to miR-335, antagomir to miR-1973, antagomir to miR-21, antagomir to miR-26a, antagomir to miR-16, premir of miR-128, premir of miR-720, premir of miR-939, premir of miR-885-3p, premir of miR-154, and premir of miR-373. For autologous stem cell therapy, the plurality of cells are isolated from a subject and have increased angiogenic function compared to the same cells (same types of cells) isolated from the subject that were not transduced with the at least one nucleic acid. The plurality of cells can include at least one of: CD34+ EPCs, MSCs, Lin− cells from bone marrow or peripheral blood, mononuclear cells from bone marrow or peripheral blood, myofibroblasts, skeletal myocytes, cardiac myocytes, satellite cells, and stem cells. In one embodiment, the at least one nucleic acid encodes antagomirs to miR-16, miR-21 and miR-92a. In another embodiment, the at least one nucleic acid encodes at least one premir of at least one miR selected from the group consisting of: miR-128, miR-720, miR-939, miR-885-3p, miR-154, and miR-373.

Further described herein is a method of promoting angiogenic function in a plurality of at least one of: stem or somatic cells with pro-angiogenic potential and progenitor cells with pro-angiogenic potential. The method includes introducing into the plurality of cells at least one nucleic acid encoding at least one of: antagomir to miR-493, antagomir to miR-515-5p, antagomir to miR-196b, antagomir to miR-1913, antagomir to miR-520a, antagomir to miR-1281, antagomir to miR-373, antagomir to miR-1978, antagomir to miR-155, antagomir to miR-92a, antagomir to miR-335, antagomir to miR-1973, antagomir to miR-21, antagomir to miR-26a, antagomir to miR-16, premir of miR-128, premir of miR-720, premir of miR-939, premir of miR-885-3p, premir of miR-154, and premir of miR-37. For use in autologous stem cell therapy, the plurality of cells are isolated from a human subject and have increased angiogenic function compared to the same cells isolated from the human subject into which the at least one nucleic acid was not introduced. The plurality of cells can include at least one of: CD34+ EPCs, MSCs, Lin− cells from bone marrow or peripheral blood, mononuclear cells from bone marrow or peripheral blood, myofibroblasts, skeletal myocytes, cardiac myocytes, satellite cells, and stem cells. In one embodiment, the at least one nucleic acid encodes antagomirs to miR-16, miR-21 and miR-92a. In another embodiment, the at least one nucleic acid encodes at least one premir of at least one miR selected from the group consisting of: miR-128, miR-720, miR-939, miR-885-3p, miR-154, and miR-373.

Yet further described herein is a method for stimulating angiogenic functions of a plurality of at least one of: stem or somatic cells with pro-angiogenic potential and progenitor cells with pro-angiogenic potential, before transplantation into a subject. The method includes the steps of: contacting the plurality of cells ex vivo with at least a first composition including at least one nucleic acid encoding at least one of: antagomir to miR-493, antagomir to miR-515-5p, antagomir to miR-196b, antagomir to miR-1913, antagomir to miR-520a, antagomir to miR-1281, antagomir to miR-373, antagomir to miR-1978, antagomir to miR-155, antagomir to miR-92a, antagomir to miR-335, antagomir to miR-1973, antagomir to miR-21, antagomir to miR-26a, antagomir to miR-16, premir of miR-128, premir of miR-720, premir of miR-939, premir of miR-885-3p, premir of miR-154, and premir of miR-37, in a suitable cell culture medium, and harvesting the plurality of cells for transplantation in a suitable medium. In the method, the plurality of cells can include at least one of: MSCs, mononuclear cells, Lin− bone marrow or peripheral blood cells, mononuclear hematopoietic stem cells, CD34+ EPCs, myofibroblasts, skeletal myocytes, cardiac myocytes, satellite cells, and stem cells. In one embodiment, the at least one nucleic acid encodes antagomirs to miR-16, miR-21 and miR-92a. In another embodiment, the at least one nucleic acid encodes at least one premir of at least one miR selected from the group consisting of: miR-128, miR-720, miR-939, miR-885-3p, miR-154, and miR-373.

Additionally described herein is a method of cell transplantation. The method includes the steps of: obtaining a plurality of cells from a subject having ischemia or ischemia-related disease; providing the cells ex vivo with conditions for cell proliferation and introducing into the cells at least one nucleic acid encoding at least one of: antagomir to miR-493, antagomir to miR-515-5p, antagomir to miR-196b, antagomir to miR-1913, antagomir to miR-520a, antagomir to miR-1281, antagomir to miR-373, antagomir to miR-1978, antagomir to miR-155, antagomir to miR-92a, antagomir to miR-335, antagomir to miR-1973, antagomir to miR-21, antagomir to miR-26a, antagomir to miR-16, premir of miR-128, premir of miR-720, premir of miR-939, premir of miR-885-3p, premir of miR-154, and premir of miR-37, resulting in a plurality of transduced cells; and transplanting the plurality of transduced cells into the subject. In the method, the plurality of transduced cells can include at least one of hematopoietic stem cells and progenitor cells. In one embodiment, the at least one nucleic acid encodes antagomirs to miR-16, miR-21 and miR-92a. In another embodiment, the at least one nucleic acid encodes at least one premir of at least one miR selected from the group consisting of: miR-128, miR-720, miR-939, miR-885-3p, miR-154, and miR-373.

Yet further described herein is a method of treating PAD or CAD in a subject. The method includes the steps of: providing a therapeutically effective amount of a composition including a plurality of at least one of: stem or somatic cells with pro-angiogenic potential and progenitor cells with pro-angiogenic potential, transduced with at least one nucleic acid encoding at least one of: antagomir to miR-493, antagomir to miR-515-5p, antagomir to miR-196b, antagomir to miR-1913, antagomir to miR-520a, antagomir to miR-1281, antagomir to miR-373, antagomir to miR-1978, antagomir to miR-155, antagomir to miR-92a, antagomir to miR-335, antagomir to miR-1973, antagomir to miR-21, antagomir to miR-26a, antagomir to miR-16, premir of miR-128, premir of miR-720, premir of miR-939, premir of miR-885-3p, premir of miR-154, and premir of miR-373; and administering the composition to the subject under conditions such that regeneration of vasculature in one or more areas of ischemia is increased in the subject. In the composition, the plurality of cells are isolated from a subject and have increased angiogenic function compared to the same cells (same types of cells) isolated from the subject that were not transduced with the at least one nucleic acid. The method can further include the step of pre-incubating the plurality of cells in a cell repair-promoting or cell survival-promoting medium including serum from healthy humans or animals, and at least one pro-survival factor, and optionally at least one anti-oxidant, for a time period of between five minutes and 5 days. In this embodiment, the plurality of cells can be pre-incubated prior to transduction with the at least one nucleic acid, concomitant with transduction with the at least one nucleic acid, or subsequent to transduction with the at least one nucleic acid. The plurality of cells can include at least one of: CD34+ EPCs, MSCs, Lin− cells from bone marrow or peripheral blood, mononuclear cells from bone marrow or peripheral blood, myofibroblasts, skeletal myocytes, cardiac myocytes, satellite cells, and stem cells.

Still further described herein is a kit for treating ischemia or ischemia-related disease in a mammalian subject. The kit includes: a therapeutically effective amount of a composition including a plurality of at least one of: stem or somatic cells with pro-angiogenic potential and progenitor cells with pro-angiogenic potential, transduced with at least one nucleic acid encoding at least one of: antagomir to miR-493, antagomir to miR-515-5p, antagomir to miR-196b, antagomir to miR-1913, antagomir to miR-520a, antagomir to miR-1281, antagomir to miR-373, antagomir to miR-1978, antagomir to miR-155, antagomir to miR-92a, antagomir to miR-335, antagomir to miR-1973, antagomir to miR-21, antagomir to miR-26a, antagomir to miR-16, premir of miR-128, premir of miR-720, premir of miR-939, premir of miR-885-3p, premir of miR-154, and premir of miR-373; and instructions for use. The plurality of cells have increased angiogenic function compared to the same cells that were not transduced with the at least one nucleic acid. The kit can be used for methods of allogeneic stem cell treatment. The subject can be a human and the ischemia or ischemia-related disease can be one or more of: atherosclerosis, CAD, PAD, acute myocardial infarction (AMI), and stroke.

Unless otherwise defined, all technical terms used herein have the same meaning as commonly understood by one of ordinary skill in the art to which this invention belongs.

As used herein, a “nucleic acid” or a “nucleic acid molecule” means a chain of two or more nucleotides such as RNA (ribonucleic acid) and DNA (deoxyribonucleic acid), and chemically-modified nucleotides. A “purified” nucleic acid molecule is one that is substantially separated from other nucleic acid sequences in a cell or organism in which the nucleic acid naturally occurs (e.g., 30, 40, 50, 60, 70, 80, 90, 95, 96, 97, 98, 99, 100% free of contaminants). The terms include, e.g., a recombinant nucleic acid molecule incorporated into a vector, a plasmid, a virus, or a genome of a prokaryote or eukaryote. Examples of purified nucleic acids include cDNAs, micro-RNAs, fragments of genomic nucleic acids, nucleic acids produced polymerase chain reaction (PCR), nucleic acids formed by restriction enzyme treatment of genomic nucleic acids, recombinant nucleic acids, and chemically synthesized nucleic acid molecules. A “recombinant” nucleic acid molecule is one made by an artificial combination of two otherwise separated segments of sequence, e.g., by chemical synthesis or by the manipulation of isolated segments of nucleic acids by genetic engineering techniques.

By the term “gene” is meant a nucleic acid molecule that codes for a particular protein, or in certain cases, a functional or structural RNA molecule.

As used herein, the phrase “transduced with at least one nucleic acid” means any method of transferring a nucleic acid into a cell; such methods include but are not necessarily limited to transfer of naked DNA in the form of oligonucleotides with or without chemical modifications and with or without optimized delivery systems for oligonucleotides including calcium phosphate, lipids (e.g., liposomes, lipifectin reagents), nanoparticles, etc. Transferring a nucleic acid into a cell can occur after cloning of a nucleic acid into plasmid or viral vectors, the latter to include, for example, AAV, adenovirus and all categories of retrovirus (e.g., lentivirus, HIV and related viruses). “Transduction” can also be used to refer to cells that have been infected with a virus (virions, particles) that contains a vector including a nucleic acid sequence to be transferred into the cell.

By the terms “microRNA,” “miRNA” and “miR” are meant short (average 22 nucleotides) non-coding RNAs. MiRs are post-transcriptional regulators that bind to complementary sequences on target messenger RNA transcripts and usually repress translational or cause mRNA target degradation with gene silencing. MiRs may be endogenous or synthetic.

As used herein, the term “antagomir” encompasses single stranded, double stranded, partially double stranded and hairpin structured chemically modified oligonucleotides that suppress (knockdown) a microRNA in a sequence-dependent manner. Antagomirs include antisense RNAs that irreversibly bind the specific miR target thereby inactivating the specific miR target with or without chemical modifications designed to improve stability. An antagomir can be referred to as a micro-RNA antagonist. Antagomirs typically are small synthetic RNAs that are complementary to the specific miR target with either mispairing at the cleavage site of Ago2 or a base modification to inhibit Ago2 cleavage. Usually, antagomirs (and premirs) are modified to make them more resistant to degradation. Pre-miRs are the same except with sense sequence to augment the levels of the target miR; premirs have the same sense sequence to endogenous miRs (while antagomirs are antisense). Pre-miRs are precursor miRs that boost miR expression. MiRs and antagomirs can be delivered as naked oligonucleotides or after cloning usually into viral vectors (e.g., isolated, cloned, etc.).

When referring to an amino acid residue in a peptide, oligopeptide or protein, the terms “amino acid residue”, “amino acid” and “residue” are used interchangably and, as used herein, mean an amino acid or amino acid mimetic joined covalently to at least one other amino acid or amino acid mimetic through an amide bond or amide bond mimetic.

As used herein, “protein” and “polypeptide” are used synonymously to mean any peptide-linked chain of amino acids, regardless of length or post-translational modification, e.g., glycosylation or phosphorylation.

When referring to a nucleic acid molecule or polypeptide, the term “native” refers to a naturally-occurring (e.g., a wild-type (WT)) nucleic acid or polypeptide.

As used herein, the phrase “sequence identity” means the percentage of identical subunits at corresponding positions in two sequences (e.g., nucleic acid sequences, amino acid sequences) when the two sequences are aligned to maximize subunit matching, i.e., taking into account gaps and insertions. Sequence identity can be measured using sequence analysis software (e.g., Sequence Analysis Software Package from Accelrys CGC, San Diego, Calif.).

The phrases “isolated” or biologically pure” refer to material (e.g., nucleic acids) which is substantially or essentially free from components which normally accompany it as found in its native state.

The term “labeled,” with regard to a nucleic acid, protein, probe or antibody, is intended to encompass direct labeling of the nucleic acid, protein, probe or antibody by coupling (i.e., physically or chemically linking) a detectable substance (detectable agent) to the nucleic acid, protein, probe or antibody.

By the term “progenitor cell” is meant any somatic cell which has the capacity to generate fully differentiated, functional progeny by differentiation and proliferation. In another embodiment, progenitor cells include progenitors from any tissue or organ system, including, but not limited to, blood, nerve, muscle, skin, gut, bone, kidney, liver, pancreas, thymus, and the like. Progenitor cells are distinguished from “differentiated cells,” which are defined in another embodiment, as those cells which may or may not have the capacity to proliferate, i.e., self-replicate, but which are unable to undergo further differentiation to a different cell type under normal physiological conditions. In one embodiment, progenitor cells are further distinguished from abnormal cells such as cancer cells, especially leukemia cells, which proliferate (self-replicate) but which generally do not further differentiate, despite appearing to be immature or undifferentiated.

As used herein, the term “totipotent” means an uncommitted progenitor cell such as embryonic stem cell, i.e., both necessary and sufficient for generating all types of mature cells. Progenitor cells which retain a capacity to generate all pancreatic cell lineages but which cannot self-renew are termed “pluripotent.” In another embodiment, cells which can produce some but not all endothelial lineages and cannot self-renew are termed “multipotent”.

As used herein, the phrases “bone marrow-derived progenitor cells” and “BM-derived progenitor cells” mean progenitor cells that come from a bone marrow stem cell lineage. Examples of bone marrow-derived progenitor cells include bone marrow-derived MSCs (BM-derived MSCs) and EPCs.

By the phrase pro-survival factor” is meant any gene product that confers cell growth and/or survival when expressed in a target tissue. Examples of pro-survival factors include VEGF and IGF-1.

The term “homing” refers to the signals that attract and stimulate the cells involved in healing to migrate to sites of injury (e.g., to ischemic areas) and aid in repair (e.g, promote regeneration of vasculature).

As used herein, the term “proangiogenic potential” means the potential of a cell or procedure to induce or enhance angiogenesis, vasculogenesis and/or arteriogenesis in any target tissue.

By the phrases “therapeutically effective amount” and “effective dosage” is meant an amount sufficient to produce a therapeutically (e.g., clinically) desirable result; the exact nature of the result will vary depending on the nature of the disorder being treated. The compositions described herein can be administered from one or more times per day to one or more times per week. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions and cells described herein can include a single treatment or a series of treatments.

As used herein, the term “treatment” is defined as the application or administration of a therapeutic agent (e.g., cells, a composition) described herein, or identified by a method described herein, to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

The terms “patient” “subject” and “individual” are used interchangeably herein, and mean a mammalian subject to be treated, with human patients being preferred. In some cases, the methods described herein find use in experimental animals, in veterinary applications, and in the development of animal models for disease, including, but not limited to, rodents including mice, rats, and hamsters, as well as non-human primates.

Although compositions, cells, kits and methods similar or equivalent to those described herein can be used in the practice or testing of the present invention, suitable compositions, cells, kits and methods are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1 is a pair of micro-RNA and gene expression (Affymetrix) array heatmaps. Arrays were performed on RNA pooled from CD34⁺/Lin⁻ EPCs of 5 subjects per group. MiR targets were computed from 4 data bases cross-linked to gene expression. Targets of selected miRs are connected; arrows indicate putative angiogenesis-related, proliferation and cell survival miRs and genes. All linked sets except BMPR2 were confined by PCR.

FIG. 2 is a series of photographs of Western blots showing CAD-related miR targets. Western blots of HUVEC proteins after transfection with the indicated premirs (+) or antagomirs (−); controls not shown.

FIG. 3 is a series of micrographs of cells showing CAD-related miR disrupted functions. (left) Representative HUVEC tubes transfected with the indicated premirs and antagomirs. (right) Incorporation of DiI-labeled Lin− cells into HUVEC (calcein-AM) tubes with (+) or without (−) antagomir 16-21-92a transfection.

FIG. 4 is a series of micrographs of cells showing control of angiogenic functions by micro-RNA and rescue with antagomirs I. Representative tube assays with calcein-AM-labeled HUVECs and DiI-labeled EPCs transfected with the indicated premirs or antagomirs. Left and right, incorporation of DiI-labeled Lin⁻ cells into HUVEC with premirs or antagomirs respectively.

FIG. 5 is a schematic illustration and a graph showing control of angiogenic functions by micro-RNA and rescue with antagomirs II. Modified Boden Chamber assays of HUVECs transfected with premirs (+) or antagomirs (−) as indicated. Premirs decreased migration whereas antagomirs increased migration of cells towards VEGF, placed in the lower chamber. Control miR-1 premir or antagomir did not effect migration.

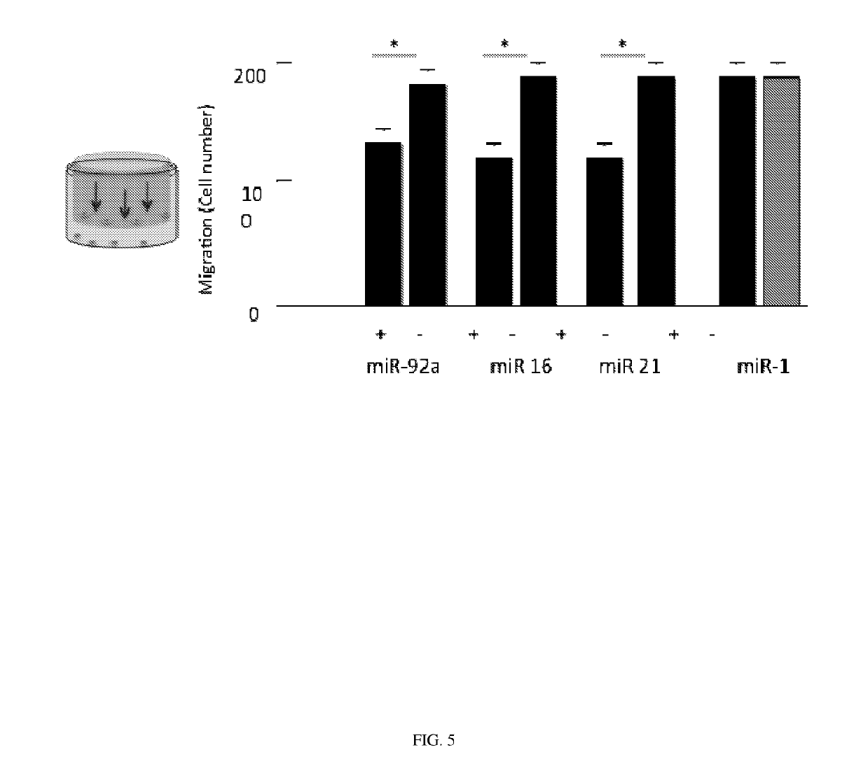

### DETAILED DESCRIPTION

The compositions, cells, methods and kits described herein may be used for treating ischemia and ischemia-related diseases in a subject, and are based on the discovery that micro-RNA expression is selectively and dramatically altered in EPCs from patients with CAD. It is proposed that the defective function of EPCs has precluded optimal stem cell therapy in all PAD and AMI trials to date. MicroRNAs (miRs) are endogenous non-coding ˜22 nucleotide RNAs that regulate the gene expression of up to 30% of the genome (Kim, Nature reviews. 2005; 6(5):376-385). The miRNA database currently lists 718 human miR sequences but the number of validated targets remains small.

In the experiments described below, the first ever evidence that micro-RNA expression is selectively and dramatically altered in EPCs from patients with CAD is presented. Many of these micro-RNAs target angiogenesis-associated genes and genes responsible for stem cell survival, proliferation and self-renewal. Transfection of selected CAD-related micro-RNAs into human endothelial cells inhibited endothelial tube formation, blocked migration to chemo-attractant cytokines and reduced proliferation and survival. Conversely, transfection of antagomirs to the same micro-RNAs improved EC function. Described herein is the use of premirs and antagomirs of micro-RNAs and combinations of microRNAs identified to be dysregulated in CAD patients to recover the function of stem cells and/or progenitor cells (e.g., EPCs), and the use of such engineered cells in the treatments of ischemia disease and ischemia-related disease such as PAD and CAD.

The below described preferred embodiments illustrate adaptations of these compositions, cells, kits and methods. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Conventional methods of gene transfer and gene therapy may also be adapted for use in the present invention. See, e.g., Gene Therapy Principles and Applications, ed. T. Blackenstein, Springer Verlag, 1999; and Gene Therapy Protocols (Methods in Molecular Medicine), ed. P. D. Robbins, Humana Press, 1997. Methods for culturing stem cells, progenitor cells and hematopoietic cells and for autologous progenitor/stem cell therapy are well known to those skilled in the art. See, e.g., Progenitor Cell Therapy for Neurological Injury (Stem Cell Biology and Regenerative Medicine), Charles S. Cox, ed., Humana Press, 1^{st} ed., 2010; A Manual for Primary Human Cell Culture (Manuals in Biomedical Research), Jan-Thorsten Schantz and Kee Woei Ng, World Scientific Publishing Co., 2^{nd} ed., 2010; and U.S. Pat. Nos. 7,790,458, 7,655,225, and 7,799,528. Design and use of antagomirs are described in, for example, U.S. patent application Ser. Nos. 12/787,552 and 12/714,863.

### Compositions For Treating Ischemia

Compositions for promoting angiogenic function in progenitor and/or stem cells (e.g., promoting recovery of function of EPCs after an ischemic event) and treating ischemia or ischemia-related disease in a subject are described herein. The compositions described herein can be used for treating any type of ischemia or ischemia-related disease or disorder, such as CAD, PAD, wound healing, kidney, liver, intestinal, scalp, brain, lung ischemia, stroke, small vessel ishemic disease, subcortical ischemic disease, ischemic cerebrovascular disease, ischemic bowel disease, carotid artery disease, ischemic colitis, diabetic retinopathy, and multiple transplanted organs including liver, kidney, heart, lung, pancreatic islets. Such compositions generally include progenitor and/or stem cells (e.g., CD34+ cells) transduced (e.g., transfected, infected, etc.) with at least one nucleic acid (e.g., one, two, three, four, etc.) encoding antagomirs to one or more (e.g., one, two, three, four, five, etc.) miRs associated with ischemic disease or ischemic-related disease, or encoding a premiR specific for one or more miRs associated with ischemic disease and/or ischemic-related disease. In a typical embodiment, cells are transduced (e.g., transfected) with antagomirs to one or more of: miR-493, miR-515-5p, miR-196b, miR-1913, miR-520a, miR-1281, miR-373, miR-1978, miR-155, miR-92a, miR-335, miR-1973, miR-21, miR-26a and miR-16; or pre-miRs of one or more of miR-128, miR-720, miR-939, miR-885-3p, miR-154, and miR-373. In some embodiments, cells may be infected with a recombinant virus containing a nucleic acid encoding antagomirs or premirs to one or more (e.g., one, two, three, four, five, etc.) miRs associated with ischemic disease or ischemic-related disease. The progenitor and/or stem cells (e.g., CD34+ cells) are cells isolated from a subject having an ischemic disease or ischemic-related disease, and they have increased angiogenic function compared to progenitor and/or stem cells (e.g., CD34+ cells) isolated from the subject that were not transfected with the at least one nucleic acid (or infected with the recombinant virus). In some embodiments, a nucleic acid that encodes at least one antagomir to a first miR and at least one premir to a second miR can be introduced into a cell for promoting angiogenic function in the cell. In an alternative embodiment, a first nucleic acid that encodes at least one antagomir to a first miR and a second nucleic acid that encodes at least one premir to a second miR can be introduced into a cell for promoting angiogenic function in the cell. In this embodiment, the first and second nucleic acids can be contained within a single vector (e.g., plasmid, viral vector) or within separate vectors. In addition to or alternative to antagomirs, any suitable molecule or reagent for decreasing or downregulating miR activity and/or expression in cells may be used. Similarly, in addition to or alternative to premirs, any suitable molecule or reagent for increasing or upregulating miR activity and/or expression in cells may be used. These may include subjection of cells to conditions that reverse the effects of a CAD-like environment in vivo that is known to include inflammation and associated oxidative stress. Methods to do this could include subjection of cells to normal serum from young healthy adults that does not contain inflammatory mediatore. The serum may also contain anti-oxidants and/or anti-inflammatory agents. In addition, once the molecular mechanism of dysregulation of the miRs is determined, methods could be devised to mimic or block these pathways. For example, it may be possible to block expression of miR-155 by incubating with IL-10 inhibitors of NF-kb, or other anti-inflammatory agents. Similarly miRs that are positively-regulated by hypoxia could be blocked by incubation in highly oxygenated medium

Any appropriate progenitor and/or stem cells can be obtained from a subject and treated as described herein for ex vivo therapy. Examples of such cells include autologous somatic cells, autologous mesenchymal stem cells from multiple sources and any other autologous stem cells including all bone marrow and peripheral blood derived Lin-negative cells as well as total mononuclear cells. Adult stem/progenitor cells may be obtained directly from the bone marrow (for example, from posterior iliac crests), any other tissue, or from peripheral blood. Isolated stem cells and progenitor cells can be maintained and propagated in a cell culture growth medium. Standardized procedures for the isolation, enrichment and storage of stem/progenitor cells are well known in the art. Methods for culturing stem cells, progenitor cells, and hematopoietic cells are well known to those skilled in the art.

The cells which are employed may be fresh, frozen, or have been subjected to prior culture. They may be fetal, neonate, adult. Hematopoietic cells may be obtained from fetal liver, bone marrow, blood, cord blood or any other conventional source. The progenitor and/or stem cells can be separated from other cells of the hematopoietic or other lineage by any suitable method.

Marrow samples may be taken from patients with ischemic disease (e.g., CAD, PAD), and enriched populations of hematopoietic stem and/or progenitor cells isolated by any suitable means (e.g., density centrifugation, counterflow centrifugal elutriation, monoclonal antibody labeling and fluorescence activated cell sorting). The stem and/or progenitor cells in this cell population can then be transfected with at least one nucleic acid (e.g., one, two, three, four, etc.) encoding antagomirs to one or more (e.g., one, two, three, four, five, etc.) miRs (e.g., one or more of: miR-493, miR-515-5p, miR-196b, miR-1913, miR-520a, miR-1281, miR-373, miR-1978, miR-155, miR-92a, miR-335, miR-1973, miR-21, miR-26a and miR-16) or premirs (e.g., one or more of miR-128, miR-720, miR-939, miR-885-3p, miR-154, and miR-373) in vitro or ex vivo and can serve as an autologous cellular therapy for ischemia (e.g., diseases associated with ischemia such as PAD and CAD). Similarly, the stem and/or progenitor cells in this cell population can instead be infected with a recombinant virus containing at least one nucleic acid (e.g., one, two, three, four, etc.) encoding antagomirs to one or more (e.g., one, two, three, four, five, etc.) miRs (e.g., three or more of: miR-493, miR-515-5p, miR-196b, miR-1913, miR-520a, miR-1281, miR-373, miR-1978, miR-155, miR-92a, miR-335, miR-1973, miR-21, miR-26a and miR-16) or premirs (e.g., one or more of miR-128, miR-720, miR-939, miR-885-3p, miR-154, and miR-373) in vitro or ex vivo and can serve as an autologous cellular therapy for ischemia (e.g., diseases associated with ischemia such as PAD and CAD).

### Methods of Autologous Progenitor/Stem Cell Therapy

Methods of autologous progenitor/stem cell therapy are described herein. Examples of such therapeutic methods include methods of treating PAD or CAD in a subject. One embodiment of a method of treating PAD or CAD in a subject includes providing a therapeutically effective amount of a composition including progenitor and/or stem cells (e.g., CD34+ cells) transduced (e.g., transfected) with at least one nucleic acid encoding antagomirs to one or more (e.g., one, two, three, four, five, etc.) of: miR-493, miR-515-5p, miR-196b, miR-1913, miR-520a, miR-1281, miR-373, miR-1978, miR-155, miR-92a, miR-335, miR-1973, miR-21, miR-26a and miR-16, or premirs specific to one or more of miR-128, miR-720, miR-939, miR-885-3p, miR-154, and miR-373; and administering the composition to the subject under conditions such that regeneration of vasculature in one or more areas of ischemia is increased in the subject. The progenitor and/or stem cells (e.g., CD34+ cells) isolated from a subject and having increased angiogenic function compared to progenitor and/or stem cells (e.g., CD34+ cells) isolated from the subject that were not transfected with the at least one nucleic acid. In some methods, a plurality of bone marrow-derived progenitor cells or stem cells are administered to the subject in an amount effective to promote regeneration of vasculature in one or more areas of ischemia in the subject. In such an embodiment, the bone marrow-derived progenitor cells or stem cells have been transduced (e.g., transfected) with at least one (e.g., one, two, three) nucleic acid encoding antagomirs to one or more (e.g., one, two, three, four, five, six, etc.) of: miR-493, miR-515-5p, miR-196b, miR-1913, miR-520a, miR-1281, miR-373, miR-1978, miR-155, miR-92a, miR-335, miR-1973, miR-21, miR-26a and miR-16, or premiRs specific to one or more of miR-128, miR-720, miR-939, miR-885-3p, miR-154, and miR-373.

In these methods, the at least one nucleic acid can be introduced into the progenitor and/or stem cells (e.g., CD34+ cells) by any suitable method or route. In a typical embodiment, the at least one nucleic acid is delivered to the targeted progenitor or stem cells by introduction of naked chemically modified oligonucleotides with or without lipofection or similar reagent, and with or without nanoparticles, and with or without a tissue targeting tag; or by cloning into an exogenous nucleic acid expression vector before delivery into the cells. Many vectors useful for transferring exogenous genes into target mammalian cells are available. The vectors may be episomal, e.g. plasmids, virus derived vectors such cytomegalovirus, adenovirus, AAV, lentivirus etc., or may be integrated into the target cell genome, through homologous recombination or random integration, e.g. retrovirus derived vectors such MMLV, HIV-1, ALV, etc. The at least one nucleic acid can be included within a viral vector, for example. Various techniques using viral vectors for the introduction of nucleic acids (e.g., antagomirs or premiRs) into cells are provided for according to the compositions and methods described herein. Viruses are naturally evolved vehicles which efficiently deliver their genes into host cells and therefore are desirable vector systems for the delivery of therapeutic nucleic acids. Preferred viral vectors exhibit low toxicity to the host cell and produce/deliver therapeutic quantities of the nucleic acid of interest (in some embodiments, in a tissue-specific manner). Retrovirus based vectors have been shown to be particularly useful when the target cells are hematopoietic stem cells. For example, see Baum et al. (1996) J Hematother 5(4):323-9; Schwarzenberger et al. (1996) Blood 87:472-478; Nolta et al. (1996) P.N.A.S. 93:2414-2419; and Maze et al. (1996) P.N.A.S. 93:206-210. Lentivirus vectors have also been described for use with hematopoietic stem cells, for example see Mochizuki et al. (1998) J Virol 72(11):8873-83. The use of adenovirus based vectors with hematopoietic cells has also been published, see Ogniben and Haas (1998) Recent Results Cancer Res 144:86-92. Viral vector methods and protocols are reviewed in Kay et al. Nature Medicine 7:33-40, 2001. Various techniques known in the art may be used to transfect the target cells, e.g. electroporation, calcium precipitated DNA, fusion, transfection, lipofection and the like.

Also in these methods, the composition or cells can be administered to a subject by any suitable route, e.g., intravenously, or directly to a target site. Several approaches may be used for the introduction of progenitor and/or stem cells (e.g., CD34+ EPCs) into the subject, including catheter-mediated delivery I.V. (e.g., endovascular catheter), or direct injection into a target site. Techniques for the isolation of autologous stem cells or progenitor cells and transplantation of such isolated cells are known in the art. Ex vivo delivery of cells transduced with nucleic acids (e.g., vectors, plasmids, etc.) encoding antagomirs and/or premiRs or with recombinant viruses expressing antagomirs and/or premiRs is encompassed by the methods described herein. Ex vivo gene delivery is used to transplant, for example, host cells (e.g., EPCs) that have been transfected with antagomirs and/or premiRs or transduced with recombinant viral vectors encoding antagomirs and/or premiRs back into the host. A suitable ex vivo protocol may include several steps. For example, a segment of target tissue (e.g., BM-derived EPCs) may be harvested from the host and an appropriate vector may be used to transduce an antagomir-encoding nucleic acid into the subject's (i.e., host's) cells. These genetically modified cells may then be transplanted back into the subject. Several approaches may be used for the reintroduction of cells into the subject, including intravenous injection, intraperitoneal injection, or in situ injection into target tissue. Microencapsulation of cells transduced or infected with recombinant viral vectors modified ex vivo, for example, is another technique that may be used. Autologous as well as allogeneic cell transplantation may be used according to the invention.

The therapeutic methods described herein in general include administration of a therapeutically effective amount of the compositions or cells described herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider. The methods and compositions herein may be also used in the treatment of any other disorders in which downregulation or upregulation of microRNAs may be implicated.

In one embodiment, a method of treating ischemia or ischemia-related disease (e.g., PAD or CAD) in a subject includes monitoring treatment progress. Monitoring treatment progress in a subject generally includes determining a measurement of, for example, vasculogenesis, vasculature, or tissue damage at the site of injury (ischemic injury) or other diagnostic measurement in a subject having, for example, CAD or PAD, prior to administration of a therapeutic amount of a composition as described herein sufficient to increase vasculogenesis at the site of injury in the subject. At one or more time points subsequent to the subject having been administered a therapeutic amount of a composition as described herein sufficient to increase vasculogenesis at the site of injury, a second measurement of vasculogenesis, vasculature or tissue damage at the site of injury is determined and compared to the first measurement of vasculogenesis, vasculature or tissue damage. The first and subsequent measurements are compared to monitor the course of PAD or CAD and the efficacy of the therapy.

In some methods of autologous progenitor/stem cell therapy, progenitor and/or stem cells transduced with the compositions for promoting angiogenic function can be transplanted into a subject who has received or concomitantly receives one or more agents that promote homing of the transduced cells to a site of ischemic injury. For example, the subject receiving the transduced progenitor and/or stem cells can be injected with a chemoattractant. The chemoattractant may be injected directly into the site of ischemic injury. Examples of chemoattractants include SDF-1, HGF, VEGF, MCP-1, and all angiogenic/vasculogenic C/CC/CXC chemokines (e.g. IL-8). The chemoattractant can be administered to the subject prior to transplantation of the transduced progenitor and/or stem cells, concomitant with transplantation of the transduced progenitor and/or stem cells, subsequent to transplantation of the transduced progenitor and/or stem cells, or at one or more of these timepoints.

### Kits

Described herein are kits for treating ischemia or ischemia-related disease (e.g., PAD or CAD) in a mammalian subject. A typical kit includes a therapeutically effective amount of a composition including progenitor and/or stem cells (e.g., CD34+ cells) transduced (e.g., transfected) with at least one (e.g., one, two, three, etc.) nucleic acid encoding antagomirs to one or more (e.g., one, two, three, four, five, etc.) miRs (e.g., one or more of: miR-493, miR-515-5p, miR-196b, miR-1913, miR-520a, miR-1281, miR-373, miR-1978, miR-155, miR-92a, miR-335, miR-1973, miR-21, miR-26a and miR-16); and/or premirs to one or more miRs (e.g., one or more of miR-128, miR-720, miR-939, miR-885-3p, miR-154, and miR-373) with instructions for administering the cells to the subject. The cells can be packaged by any suitable means for transporting and storing cells; such methods are well known in the art. The instructions generally include one or more of: a description of the cells; dosage schedule and administration for treatment of ischemia and ischemia-related disorders (e.g., PAD, CAD); precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container. Generally, a kit as described herein also includes packaging. In some embodiments, the kit includes a sterile container which contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding cells or medicaments.

### Administration of Compositions

The compositions and cells described herein may be administered to mammals (e.g., rodents, humans) in any suitable formulation. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to mammals by any conventional technique. The compositions may be administered directly to a target site by, for example, surgical delivery to an internal or external target site, or by catheter (e.g., endovascular catheter) to a site accessible by a blood vessel. When treating a subject having, for example, PAD or CAD, the composition may be administered to the subject intravenously, directly into cardiovascular tissue or arterial tissue, or to the surface of cardiovascular or arterial tissue. The compositions may be administered in a single bolus, multiple injections, or by continuous infusion (e.g., intravenously, by peritoneal dialysis, pump infusion). For parenteral administration, the compositions are preferably formulated in a sterilized pyrogen-free form.

### Effective Doses

The compositions and cells described herein are preferably administered to a mammal (e.g., human) in an effective amount, that is, an amount capable of producing a desirable result in a treated mammal (e.g., treating ischemic conditions such as CAD or PAD). Such a therapeutically effective amount can be determined according to standard methods. Toxicity and therapeutic efficacy of the compositions utilized in methods of the invention can be determined by standard pharmaceutical procedures. As is well known in the medical and veterinary arts, dosage for any one subject depends on many factors, including the subject's size, body surface area, age, the particular composition to be administered, time and route of administration, general health, and other drugs being administered concurrently.

### EXAMPLES

The present invention is further illustrated by the following specific examples. The examples are provided for illustration only and should not be construed as limiting the scope of the invention in any way.

### Example 1

### Bone marrow-derived CD34⁺/Lin⁻ putative endothelial progenitor cells from patients with CAD have dysregulated expression of selected micro-RNAs and target genes

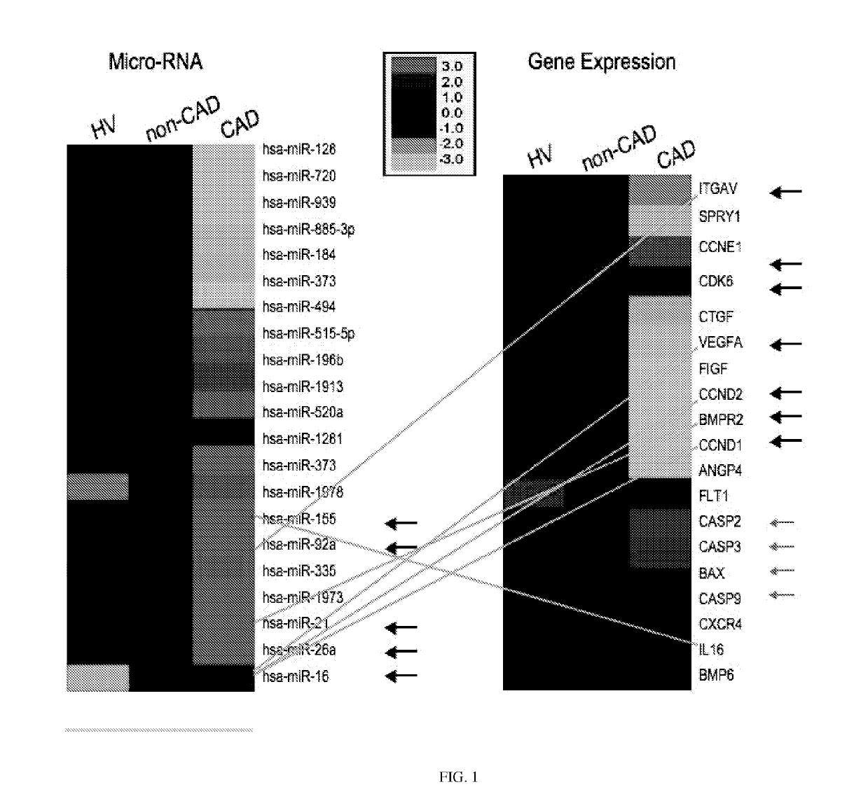

To determine the effects of CAD on molecular genetic changes in human bone marrow cells, isolated CD34⁺/Lin⁻ cells were isolated from the bone marrow of 5 patients with CAD, 5 age-matched patients undergoing cardiothoracic surgery for non-CAD related conditions, and 3 healthy volunteers. Bone marrow (5-20 mL) was collected by aspiration from the sternum into a heparin syringe during surgery. Mononuclear cells were isolated by Histopaque and the cells frozen in 50% IMDM, 40% FBS and 10% DMSO at a density of 10⁷ cells/ml. Similar yields of viable CD34⁺/Lin⁻ cells were obtained from each group. FACS analyses indicated similar cell surface profiles of cells from each group. FIG. 1 shows heatmaps of micro-RNAs (left) and genes (right) that displayed the greatest changes between groups. The blue lines join miRs with their putative target genes. Note that the main differences are seen between CAD and non-CAD groups and several of the most strongly induced miRs target genes that are required for angiogenesis, stem cell self renewal, proliferation and survival. These include integrin a-5 and -V (ITGAV/5) required for angiogenesis and vascular regeneration, cyclins D1 and D2 (CCND1 and CCND2), bone morphogenic protein receptor 2 (BMPR2), and VEGF. IL-16 (>3-fold increased), a T-cell chemotactic cytokine, may contribute to the upregulation of miR-155 (also up 3-fold). MiR-155 has multiple targets that may modulate vascular function including the Ang-II type 1 receptor, IKKe, FADD and TNF-a. MiR-155 has been shown to increase TNF-a production relieving self-inhibition by a 3′-UTRsite of TNF-a mRNA. All of the selected miRs shown in FIG. 1 were confirmed by RT-PCR to increase by 4-6-fold in the CAD group. Other dysregulated miRs of interest include miR-210 a hypoxia-regulated miR that targets mitochondrial iron-sulphur clusters and Ephrin-A3 and the angiotensin receptor-like 1 (AGTRL1). Down-regulated miRs also include miR200C that targets E-cadherin and Flt1. MiR200C was decreased >20-fold in CAD samples while Flt1 transcripts increased 20-fold (by RT-PCR). Mir-145 a stem cell differentiation miR decreased >6-fold in the CAD group. Dysregulated miRs have targets that may exert positive or negative effects on the functions of CD34+/Lin− cells in the CAD group.

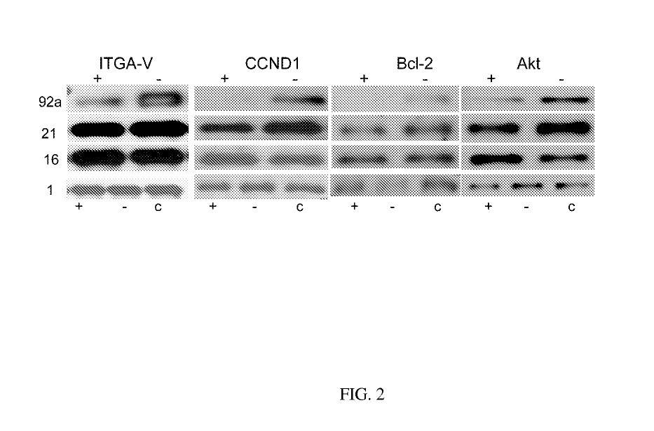

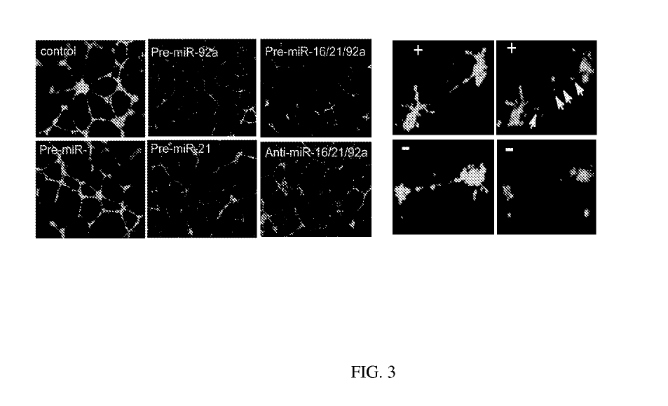

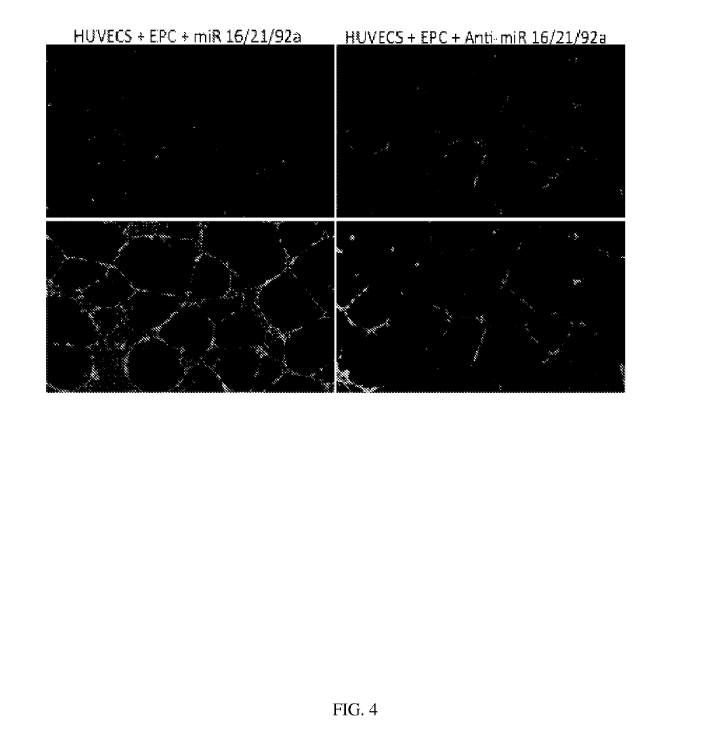

FIG. 2 describes preliminary characterizations of target proteins and functions of 3 key miRs. Expression of 4 predicted targets of miR92a, ITGA-V, CCND1, Bcl2 and Akt were all decreased in HUVECs by premir transfection (mimicking the effects of over-expression of these miRs in CAD EPCs). FIG. 3 shows representative panels of angiogenic tube assays. Infection of HUVECs with premirs-92a or -21 blocked tube formation (premir-16 had the same effect as premir-21 (not shown); miR-1 (control) did not block tube formation). MiR-92a was the most inhibitory of individual miRs buta combination of all 3 was significantly more inhibitory than any premir alone (3, top right). Individual antagomirs did not inhibit tube formation. Combined antagomirs improved tube formation as evidenced by thinner more compact tubes compared with controls or cells treated with pre-miR-1 (FIG. 3, right and middle panels). This is the first evidence that knock-down of combined anti-angiogenic micro-RNAs by transfection of antagomirs can improve the function of endothelial cells. As another means to determine whether functional recovery was possible by antagomir treatment, Lin⁻ cells from CAD patients were transfected with 16/21/92a-antagomirs, labeled with DiI and cells ±transfection added 1:3 to a modified HUVEC tube assay. It was found that Lin⁻ (orange) cells incorporated into linear tubes and cell clusters at the end of the tubes and antagomir transfections increased the incorporation of Lin− cells into the linear regions (arrows in FIG. 3 right panels and FIG. 4). Interestingly, Lin− EPCs over-expressing miRs 16/21/92a were generally inhibitory to HUVEC tube formation in this assay (FIG. 4).

In conclusion, these results indicate that elevations of miRs 16, 21 and 92a in HUVECS or CD34⁺/Lin⁻ cells of CAD patients contribute to or cause defective function whereas antagomirs promote function. Six micro-RNAs were identified that are selectively decreased in EPCs from CAD patients and 14 that are increased. Such dysregulation of these micro-RNAs contributes to CAD progression and the inability of autologous EPCs to provide optimal therapy for CAD and PAD. Thus, cells and kits for correcting function of EPCs by reversing the dysregulated micro-RNAs (e.g., by transfection) include use of premirs and antagomirs in multiple combinations.

### Other Embodiments

Any improvement may be made in part or all of the compositions, cells, kits, and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A composition comprising a plurality of at least one of: stem or somatic cells with pro-angiogenic potential and progenitor cells with pro-angiogenic potential, transduced with at least one nucleic acid encoding at least one of: antagomir to miR-493, antagomir to miR-515-5p, antagomir to miR-196b, antagomir to miR-1913, antagomir to miR-520a, antagomir to miR-1281, antagomir to miR-373, antagomir to miR-1978, antagomir to miR-155, antagomir to miR-92a, antagomir to miR-335, antagomir to miR-1973, antagomir to miR-21, antagomir to miR-26a, antagomir to miR-16, premir of miR-128, premir of miR-720, premir of miR-939, premir of miR-885-3p, premir of miR-154, and premir of miR-373;
the plurality of cells isolated from a subject and having increased angiogenic function compared to the same cells isolated from the subject that were not transduced with the at least one nucleic acid.

2. The composition of claim 1, wherein the plurality of cells comprise at least one selected from the group consisting of: CD34+ endothelial progenitor cells (EPCs), mesenchymal stem cells (MSCs), Lin− cells from bone marrow or peripheral blood, mononuclear cells from bone marrow or peripheral blood, myofibroblasts, skeletal myocytes, cardiac myocytes, satellite cells, and stem cells.

3. The composition of claim 2, wherein the subject is a human and the plurality of cells are CD34+ EPCs obtained from the human subject.

4. The composition of claim 1, wherein the at least one nucleic acid encodes antagomirs to miR-16, miR-21 and miR-92a.

5. The composition of claim 1, wherein the at least one nucleic acid encodes at least one premir of at least one miR selected from the group consisting of: miR-128, miR-720, miR-939, miR-885-3p, miR-154, and miR-373.

6. The composition of claim 1, wherein the at least one nucleic acid is a viral vector.

7. The composition of claim 1, further comprising a suitable medium for transplantation of the transduced plurality of cells into the subject.

8. A plurality of at least one of: stem or somatic cells with pro-angiogenic potential and progenitor cells with pro-angiogenic potential, each cell of the plurality of cells transduced with at least one nucleic acid encoding at least one of: antagomir to miR-493, antagomir to miR-515-5p, antagomir to miR-196b, antagomir to miR-1913, antagomir to miR-520a, antagomir to miR-1281, antagomir to miR-373, antagomir to miR-1978, antagomir to miR-155, antagomir to miR-92a, antagomir to miR-335, antagomir to miR-1973, antagomir to miR-21, antagomir to miR-26a, antagomir to miR-16, premir of miR-128, premir of miR-720, premir of miR-939, premir of miR-885-3p, premir of miR-154, and premir of miR-373;
the plurality of cells isolated from a subject and having increased angiogenic function compared to the same cells isolated from the subject that were not transduced with the at least one nucleic acid.

9. The plurality of cells of claim 8, wherein the plurality of cells comprise at least one selected from the group consisting of: CD34+ EPCs, MSCs, Lin− cells from bone marrow or peripheral blood, mononuclear cells from bone marrow or peripheral blood, myofibroblasts, skeletal myocytes, cardiac myocytes, satellite cells, and stem cells.

10. The plurality of cells of claim 8, wherein the at least one nucleic acid encodes antagomirs to miR-16, miR-21 and miR-92a.

11. The plurality of cells of claim 8, wherein the at least one nucleic acid encodes at least one premir of at least one miR selected from the group consisting of: miR-128, miR-720, miR-939, miR-885-3p, miR-154, and miR-373.

12. A method of promoting angiogenic function in a plurality of at least one of: stem or somatic cells with pro-angiogenic potential and progenitor cells with pro-angiogenic potential, comprising introducing into the plurality of cells at least one nucleic acid encoding at least one of: antagomir to miR-493, antagomir to miR-515-5p, antagomir to miR-196b, antagomir to miR-1913, antagomir to miR-520a, antagomir to miR-1281, antagomir to miR-373, antagomir to miR-1978, antagomir to miR-155, antagomir to miR-92a, antagomir to miR-335, antagomir to miR-1973, antagomir to miR-21, antagomir to miR-26a, antagomir to miR-16, premir of miR-128, premir of miR-720, premir of miR-939, premir of miR-885-3p, premir of miR-154, and premir of miR-37.

13. The method of claim 12, wherein the plurality of cells are isolated from a human subject and have increased angiogenic function compared to the same cells isolated from the human subject into which the at least one nucleic acid was not introduced.

14. The method of claim 12, wherein the plurality of cells comprise at least one selected from the group consisting of: CD34+ EPCs, MSCs, Lin− cells from bone marrow or peripheral blood, mononuclear cells from bone marrow or peripheral blood, myofibroblasts, skeletal myocytes, cardiac myocytes, satellite cells, and stem cells.

15. The method of claim 12, wherein the at least one nucleic acid encodes antagomirs to miR-16, miR-21 and miR-92a.

16. The method of claim 12, wherein the at least one nucleic acid encodes at least one premir of at least one miR selected from the group consisting of: miR-128, miR-720, miR-939, miR-885-3p, miR-154, and miR-373.

17. A method for stimulating angiogenic functions of a plurality of at least one of:
stem or somatic cells with pro-angiogenic potential and progenitor cells with pro-angiogenic potential, before transplantation into a subject, the method comprising the steps of: (i) contacting the plurality of cells ex vivo with at least a first composition comprising at least one nucleic acid encoding at least one of: antagomir to miR-493, antagomir to miR-515-5p, antagomir to miR-196b, antagomir to miR-1913, antagomir to miR-520a, antagomir to miR-1281, antagomir to miR-373, antagomir to miR-1978, antagomir to miR-155, antagomir to miR-92a, antagomir to miR-335, antagomir to miR-1973, antagomir to miR-21, antagomir to miR-26a, antagomir to miR-16, premir of miR-128, premir of miR-720, premir of miR-939, premir of miR-885-3p, premir of miR-154, and premir of miR-37, in a suitable cell culture medium, and (ii) harvesting the plurality of cells for transplantation in a suitable medium.

18. The method of claim 17, wherein the plurality of cells comprise at least one selected from the group consisting of: MSCs, mononuclear cells, Lin− bone marrow or peripheral blood cells, mononuclear hematopoietic stem cells, CD34+ EPCs, myofibroblasts, skeletal myocytes, cardiac myocytes, satellite cells, and stem cells.

19. The method of claim 17, wherein the at least one nucleic acid encodes antagomirs to miR-16, miR-21 and miR-92a.

20. The method of claim 17, wherein the at least one nucleic acid encodes at least one premir of at least one miR selected from the group consisting of: miR-128, miR-720, miR-939, miR-885-3p, miR-154, and miR-373.

21. A method of cell transplantation comprising:
(a) obtaining a plurality of cells from a subject having ischemia or ischemia-related disease;
(b) providing the cells ex vivo with conditions for cell proliferation and introducing into the cells at least one nucleic acid encoding at least one of: antagomir to miR-493, antagomir to miR-515-5p, antagomir to miR-196b, antagomir to miR-1913, antagomir to miR-520a, antagomir to miR-1281, antagomir to miR-373, antagomir to miR-1978, antagomir to miR-155, antagomir to miR-92a, antagomir to miR-335, antagomir to miR-1973, antagomir to miR-21, antagomir to miR-26a, antagomir to miR-16, premir of miR-128, premir of miR-720, premir of miR-939, premir of miR-885-3p, premir of miR-154, and premir of miR-37, resulting in a plurality of transduced cells; and
(c) transplanting the plurality of transduced cells into the subject,
wherein the plurality of transduced cells comprises at least one of hematopoietic stem cells and progenitor cells.

22. The method of claim 21, wherein the at least one nucleic acid encodes antagomirs to miR-16, miR-21 and miR-92a.

23. The method of claim 21, wherein the at least one nucleic acid encodes at least one premir of at least one miR selected from the group consisting of: miR-128, miR-720, miR-939, miR-885-3p, miR-154, and miR-373.

24. A method of treating peripheral artery disease (PAD) or coronary artery disease (CAD) in a subject, the method comprising the steps of: providing a therapeutically effective amount of the composition of claim 1; and administering the composition to the subject under conditions such that regeneration of vasculature in one or more areas of ischemia is increased in the subject.

25. The method of claim 24, further comprising the step of pre-incubating the plurality of cells in a cell repair-promoting or cell survival-promoting medium comprising serum from healthy humans or animals, and at least one pro-survival factor, and optionally at least one anti-oxidant, for a time period of between five minutes and 5 days,
wherein the plurality of cells are pre-incubated prior to transduction with the at least one nucleic acid, concomitant with transduction with the at least one nucleic acid, or subsequent to transduction with the at least one nucleic acid.

26. The method of claim 24, wherein the plurality of cells comprise at least one selected from the group consisting of: CD34+ EPCs, MSCs, Lin− cells from bone marrow or peripheral blood, mononuclear cells from bone marrow or peripheral blood, myofibroblasts, skeletal myocytes, cardiac myocytes, satellite cells, and stem cells.

27. A kit for treating ischemia or ischemia-related disease in a mammalian subject, the kit comprising:
(a) a therapeutically effective amount of a composition comprising a plurality of at least one of: stem or somatic cells with pro-angiogenic potential and progenitor cells with pro-angiogenic potential, transduced with at least one nucleic acid encoding at least one of: antagomir to miR-493, antagomir to miR-515-5p, antagomir to miR-196b, antagomir to miR-1913, antagomir to miR-520a, antagomir to miR-1281, antagomir to miR-373, antagomir to miR-1978, antagomir to miR-155, antagomir to miR-92a, antagomir to miR-335, antagomir to miR-1973, antagomir to miR-21, antagomir to miR-26a, antagomir to miR-16, premir of miR-128, premir of miR-720, premir of miR-939, premir of miR-885-3p, premir of miR-154, and premir of miR-373;
the plurality of cells having increased angiogenic function compared to the same cells that were not transduced with the at least one nucleic acid; and
(b) instructions for use.

28. The kit of claim 27, wherein the subject is a human and the ischemia or ischemia-related disease is selected from the group consisting of: atherosclerosis, CAD, PAD, acute myocardial infarction (AMI), and stroke.
